(19) United States
(12) Patent Application Publication
(10) Pub. No.: US 2026/0363971 A1
(43) Pub. Date: Nov. 19, 2026

(54) WHITE COMPOSITION AND USE THEREOF IN VARIOUS FORMULATIONS, IN PARTICULAR COSMETIC FORMULATIONS

(71) Applicants: OLIKROM, PESSAC (FR); CHANEL PARFUMS BEAUTE, NEUILLY-SUR-SEINE (FR)

(72) Inventors: Jean-François LETARD, Canéjan (FR); Philippe GUARILLOFF, Pantin (FR)

(21) Appl. No.: 19/132,475

(22) PCT Filed: Nov. 24, 2023
(86) PCT No.: PCT/EP2023/082986

§ 371 Date: May 23, 2025

(30) Foreign Application Priority Data
Nov. 25, 2022 (EP) 22209568.9

(51) Int. Cl. A61K 8/22; A61K 8/19; A61K 8/27; A61Q 19/00; C09C 1/00

(52) U.S. Cl. CPC A61K 8/22; A61K 8/19; A61K 8/27; A61Q 19/00; C09C 1/0081; C09C 1/0084; A61K 2800/10; A61K 2800/262; A61K 2800/43; C01P 2006/19; C01P 2006/62

(57) ABSTRACT

The present invention relates to a composition which is intended to replace TiO2, the method for manufacturing this composition and uses thereof. In particular, the present invention relates to a water-insoluble white composition, comprising the mixture of bismuth oxychloride, boron nitride and at least one other inorganic compound, wherein the mixture of bismuth oxychloride and boron nitride is at least 50 wt. % relative to the total weight of the composition. Said composition is capable of substituting titanium dioxide and is suitable for various uses, in particular cosmetic uses. The invention also relates to the method for preparing this composition and uses thereof.

### TECHNICAL FIELD

The present invention relates to the technical field of pigments. The object of the invention is in particular a specific white composition capable of substituting titanium dioxide (TiO2), notably in cosmetics. The invention also relates to the method for preparing this composition and uses thereof.

### PRIOR ART

Pigments are fundamental in the composition of certain products to give color. They are notably used extensively in cosmetic products where they add color and opacity to finished products. In make-up products, pigments are essential because they contribute to the look of the skin or the nails. Pigments are also present in hair coloring products or else in skin care products to give a specific color to the skin.

Pigments are solid and insoluble molecules. They can be of animal, plant or synthetic origin, and can be inorganic or organic in nature.

Today, the pigments most widely used in industry are pigments of inorganic origin. Indeed, their resistance to light and their low cost make them highly sought-after materials. They consist of dry ground minerals, generally metals or metallic salts. Due to their composition, inorganic pigments are generally more opaque and more insoluble than organic pigments.

From the mineral pigments commonly used, TiO2 can in particular be cited. It is an inorganic pigment used in powder form in numerous applications for its incomparable white color and its numerous other properties such as ultraviolet absorption, its opacifying properties, its resistance to chemical products, its thermal stability and its photocatalyst potential.

By virtue of its remarkable properties, TiO2 is used in the formulation of a wide variety of finished products such as paints, inks, medicines, toothpastes, construction products (building and public works) and formulations for cosmetics products.

With the evolution of regulations, more and more studies are focusing on the harmlessness of pigments to humans and the environment. Some recent studies have demonstrated the carcinogenic potential of TiO2 by inhalation.

In the field of cosmetics, in order to avoid inhalation, TiO2 is no longer used in the form of small particle powders in certain formulations such as sprays.

Although its use in products for topical application is not prohibited, manufacturers, notably in the cosmetics industry, are still seeking new compounds to offer an alternative to the ubiquitous use of TiO2 that is suitable for use on the skin and skin appendages.

### SUMMARY OF THE INVENTION

To meet this need, the invention proposes a new composition capable of providing an alternative to TiO2, particularly suitable for use in cosmetics.

In particular, the object of the invention is therefore a water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

Advantageously, the presence of at least 50% bismuth oxychloride and boron nitride within the composition according to the invention allows the composition to exhibit clarity and opacity characteristics similar to those of titanium dioxide.

Preferentially, the composition according to the invention has a clarity defined by an L* parameter greater than 88, an opacity greater than 82%, comprising the mixture of bismuth oxychloride, boron nitride and at least one other inorganic compound.

Preferentially, the composition according to the invention is in powder form.

It has properties equivalent to those of TiO2, namely clarity a defined by an L* parameter greater than 88 and an opacity greater than 82%.

Bismuth oxychloride is a water-insoluble inorganic pigment obtainable from copper, tin or lead. It is used in numerous fields, notably in cosmetics in eyeshadows, lipstick and nail polishes. Bismuth oxychloride has an L* clarity of 85 and an opacity of 85%. Thus, the use of bismuth oxychloride alone is insufficient to constitute an alternative to TiO2, particularly in cosmetics.

Boron nitride is a white chemical compound used in numerous fields, notably in cosmetics. Due to its structural properties, boron nitride can take on a variety of shapes, notably lamellar or honeycomb. This so-called hexagonal conformation gives it a soft and silky feel improving the spreadability of the formulation it comprises, notably on the skin. Boron nitride has an L* clarity of 90 and an opacity of 86%. Thus, the use of boron nitride alone is insufficient to constitute an alternative to TiO2, in particular in cosmetics.

Advantageously, the combination of the compounds according to the invention makes it possible to generate a synergistic effect on the clarity and/or the opacity of the composition which are greater than the clarity and/or the opacity of each of the compounds making up the composition taken individually.

The composition according to the invention has a clarity that makes it suitable for use in particular in cosmetic formulations, medicines, inks and paints as a substitute for TiO2.

According to a particularly suitable embodiment, the composition according to the invention comprises the mixture of at least 4 inorganic compounds.

Preferentially, the composition according to the invention also comprises, in addition to the mixture of bismuth oxychloride and boron nitride, at least one other inorganic compound selected from iron oxide, zinc oxide, cerium oxide, calcium carbonate, tricalcium phosphate, calcium sulfate, zinc stearate, magnesium carbonate, zinc sulfate, magnesium stearate, barium sulfate, zinc sulfide, aluminum powder, calcined clay, tricalcium phosphate, kaolin and combinations thereof.

According to a preferred embodiment of the invention, the composition has an oil absorption of less than 55%.

Such a composition is particularly useful for providing an alternative to the use of TiO2 in various formulations and applications, in particular in cosmetics.

According to another aspect, the invention relates to a method for manufacturing a composition according to the invention comprising notably a step of selecting compounds constituting the composition according to the invention, mixing said constituents and recovering the composition according to the invention.

Finally, the invention also covers the use of the composition according to the invention in inks, paints, plastics and cosmetic product formulations.

Further features and advantages will become apparent from the detailed description of the invention and the examples, which are illustrative only and by no means limit the scope of the invention.

### DETAILED DESCRIPTION OF THE INVENTION

### Definitions

For the purposes of the invention, “clarity” means an indication of the whiteness of the color under study. The darker the color, the lower the clarity. To measure the clarity, it is necessary to deposit the composition according to the invention on black background contrast cards by forming a film of uniform thickness. The measurement is obtained using a spectrophotometer equipped with an integrating sphere and a D65 illuminant. The closer the L* parameter is to 100, the whiter the composition.

For the purposes of the invention, “opacity” means the ability of a composition to conceal a surface covered by a given amount. The opacity is measured by its hiding power. The opacity can be measured by calculating the contrast ratio on a black and white background. The composition can be applied with a film applicator to a known thickness. After a drying period, a spectrophotometer measures a reflectance value for the overlay on a white background (Yw) and on a black background (Yb). The opacity of a composition is thus measured by calculating the contrast ratio Yb/Yw which is expressed as a percentage.

For the purpose of the invention, “reflectance” means the measurement of the proportion of light reflected from a surface.

For the purposes of the invention, “oil absorption” means the amount of linseed oil (in grams) absorbed by 100 g of composition according to the invention to obtain a firm and homogeneous paste. More particularly, measuring the oil absorption according to the invention consists of mixing the composition according to the invention with the minimum amount of linseed oil to obtain a plastic mass (powder+oil) which is easily detached from the support (spatula and/or glass plate) on which the mixing is carried out. The higher this value, the more oil the composition absorbs.

For the purposes of the invention, “film-forming polymer” means a polymer capable of forming, on its own or in the presence of an auxiliary film-forming agent, a continuous and adherent film on a support, notably on keratin materials.

For the purposes of the invention, “wax” means a fatty substance with a reversible liquid/solid phase change, having a melting temperature greater than 55 °C and generally less than 110 °C, which is liquid under the conditions of preparing the formulation according to the invention and which has an anisotropic crystalline organization in the solid state. The wax can be polar or non-polar

For the purposes of the invention, “polar wax” means a wax comprising at least one heteroatom such as oxygen, nitrogen, silicon or phosphorus.

For the purpose of the invention, “non-polar wax” means a hydrocarbon wax and/or a silicone wax.

For the purposes of the invention, “filler” means any insoluble particle of any shape (notably spherical or lamellar), mineral or organic, in the formulation.

### Composition

The object of the present invention is therefore a water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

According to one embodiment of the invention, the composition according to the invention comprises a mixture of bismuth oxychloride and boron nitride representing at least 55%, preferentially at least 60%, even more preferentially at least 70%, notably 75% by weight relative to the total weight of the composition.

According to another embodiment, the composition according to the invention comprises a mixture of bismuth oxychloride and boron nitride representing between 50 and 95%, notably 65 and 95%, more preferentially between 80 and 95% by weight relative to the total weight of the composition.

Advantageously, the content of the mixture of bismuth oxychloride and boron nitride is essential to provide a clarity and an opacity equivalent to that of TiO2.

According to a particularly preferred embodiment, the composition according to the invention does not comprise TiO2.

According to another preferred embodiment, the composition according to the invention has a clarity measured on a black background defined by a L* parameter greater than 88 and an opacity greater than 82%.

Preferentially, the composition according to the invention has a clarity measured on a black background defined by a L* parameter greater than 90 and an opacity greater than 82%.

The clarity of the composition according to the invention is assessed by measuring, against a black background, the chromatic coordinate L* from the CIELAB color space, generally known as CIE 1976, using an integrating sphere spectrophotometer under D65 illumination.

Preferably, the composition according to the invention has an L* clarity greater than 90, notably greater than 91, preferentially greater than 95.

Advantageously, the higher the clarity of the composition according to the invention, the whiter the composition.

The opacity of the composition according to the invention is evaluated by measuring its hiding power using a spectrophotometer measuring a reflectance value of the composition applied on a white background (Yw) and on a black background (Yb).

Preferably, the composition according to the invention has an opacity greater than 85%, notably greater than 87%, preferentially greater than 90%, even more preferentially greater than 92%.

According to a preferred embodiment, the composition according to the invention has a clarity L* greater than 91 and/or an opacity greater than 88%. According to a particularly preferred variant, the composition according to the invention has a clarity L* greater than or equal to 92, and an opacity greater than or equal to 90%.

Preferentially, the composition according to the invention has greater clarity and/or greater opacity than each of the compounds making up the composition.

Advantageously, the selected compounds have a synergistic effect on the L* parameter measured on a black background and on the opacity.

When the composition is used in inks or paint, the composition according to the invention advantageously retains its characteristics, notably its clarity and its opacity, even after drying. After application, the composition according to the invention can form a dry film retaining its structural organization, notably its opacity and its clarity.

The composition according to the invention comprises a mixture of at least 3 inorganic compounds, preferentially at least 4, even more preferentially at least 5.

Preferably, the composition according to the invention also comprises, in addition to the mixture of bismuth oxychloride and boron nitride, at least one other inorganic compound selected from iron oxide, zinc oxide, cerium oxide, calcium carbonate, tricalcium phosphate, calcium sulfate, zinc stearate, magnesium carbonate, zinc sulfate, magnesium stearate, barium sulfate, zinc sulfide, aluminum powder, calcined clay, kaolin and combinations thereof.

According to a particularly preferred embodiment, the composition according to the invention also comprises, in addition to the mixture of bismuth oxychloride and boron nitride, at least one other inorganic compound selected from zinc stearate, zinc sulfate, zinc oxide, zinc sulfide and combinations thereof.

Preferably, the composition according to the invention comprises zinc sulfate and/or zinc oxide in addition to the mixture of bismuth oxychloride and boron nitride.

According to another preferred embodiment, the composition according to the invention has an oil absorption of less than 55%, preferentially less than 40%, notably between 25 and 55%, even more preferentially between 35 and 45%.

Advantageously, the oil absorption of the composition according to the invention facilitates its integration into cosmetic product formulations.

According to another embodiment, the composition according to the invention comprises a coating. When the composition according to the invention comprises a coating, this is selected from a hydrophobic coating, a hydrophilic coating, a lipophobic coating or a lipophilic coating.

The coating, in the context of the invention, is preferentially a coating of natural origin.

Preferentially, when the composition according to the invention comprises a coating, the latter comprises at least one molecule selected from:
a silicone derivative preferably selected from Triethoxycaprylylsilane, Dimethicone and Methicone;
a fatty acid ester derivative preferably selected from stearoyl glutamic acid, polyhydroxystearic acid, hydrogenated olive oil stearyl ester, magnesium myristate, Jojoba ester, lecithin, hydrogenated lecithin, aloe vera oil extract and avocado oil;
waxes preferably selected from carnauba wax, polyethylene wax or olefin-like waxes;
silane derivatives, preferably selected from methoxy PEG-10 propyltrimethoxysilane;
silica;
phytic acid, inositol dihydrogen phosphate ester;
metallic salts of fatty acids;
metal alcoholates;
polysaccharides;
alkyl titanate;
natural or mineral oils;
amino acids or amino acid derivatives such as lauroyl lysine alone or combined with aluminum hydroxide;
a fatty acid stearic acid derivative, preferably isopropyl titanium triisostearate; and
a fluorosilane, preferably perfluorohexylethyl triethoxysilane.

Advantageously, the coating of the composition according to the invention facilitates the dispersion of the composition in cosmetic formulations. A person skilled in the art can select the type of coating based on the nature and intended use of the formulation, notably for cosmetic use.

Preferentially, the composition according to the invention is in powder form.

The composition according to the invention is thus particularly suitable for replacing TiO2 in cosmetic product formulations.

According to another aspect, the composition according to the invention is capable of being obtained by a manufacturing method comprising the following steps:
a) Selecting each compound constituting the composition according to the invention: bismuth oxychloride, boron nitride and at least one other inorganic compound;
b) Mixing the compounds selected in step a) by successive addition;
c) Recovering a water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

The invention also relates to a formulation comprising the composition according to the invention.

Preferentially, the formulation according to the invention does not comprise titanium dioxide.

According to one embodiment, the formulation according to the invention comprises between 0.1 and 20 wt. % of composition according to the invention, preferentially between 0.5 and 10% and even more preferentially between 1 and 8%, relative to the total weight of the formulation.

Advantageously, the necessary amount of composition according to the invention to be added to a formulation to replace TiO2 while obtaining a clarity and an opacity similar to that of the same formulation comprising TiO2 is less than 4 times the amount of TiO2 present in the formulation, preferentially less than 3 times.

Advantageously, the amount of composition according to the invention to be added to the formulation to obtain a white color and an opacifying power similar to that of TiO2 is less than the amount of coloring matter of the prior art not presenting the desired colorimetric characteristics. However, occupying a more significant proportion in a cosmetic formulation entails additional constraints such as surface tension, texture, hardness or else oil absorption. The formulation according to the invention can comprise a cosmetically acceptable medium, that is capable of being applied to the skin, the lips or the eyelashes without generating discomfort for the user.

The formulation according to the invention can notably take the form of a mascara formulation, a lipstick or lip gloss formulation, the form of an eyeshadow and/or a cheek shadow formulation or else the form of a foundation formulation or a face or body care product.

Preferably, the formulation according to the invention can be presented in any form suitable for the field of cosmetics, notably in the form of a powder, an emulsion, a microemulsion, a nanoemulsion, a suspension, a solution, a lotion, a cream, an aqueous or hydroalcoholic gel, a foam, a serum, an aerosol solution or dispersion, or a lipid vesicle dispersion, or droplets obtained by coacervation or by microfluidics.

According to one embodiment, the formulation according to the invention is in the form of an anhydrous formulation, i.e. containing less than 5 wt. % of water, preferentially less than 1 wt. % of water.

According to another embodiment, the formulation according to the invention takes the form of a water-in-oil or oil-in-water emulsion.

The formulation according to the invention can also comprise, in addition to the composition according to the invention, at least one additive selected from an emollient agent, a humectant agent, a gelling and/or thickening agent, a film-forming agent, a surfactant, an oil, a non-volatile organic solvent, a filler, a wax, an active agent, a colorant, a preservative, an antioxidant agent, an organic or inorganic powder, a sunscreen and a fragrance.

When the formulation according to the invention comprises at least one humectant agent, at least one humectant agent is selected from:
polyols such as glycerin, diglycerin, propylene glycol, caprylyl glycol, pentylene glycol or hexanediol);
sugars;
glycosaminoglycans such as hyaluronic acid and salts and esters thereof; and
polyquaterniums such as Lipidure-PMB.

Preferably, the formulation according to the invention comprises, in addition to the composition according to the invention, at least one humectant agent having a content of between 0 and 30%, preferably between 0.005 and 10% by total weight of the formulation.

When the formulation according to the invention comprises at least one emollient agent, at least one emollient agent is selected from:
esters such as jojoba esters, fatty acid esters and fatty alcohol esters, preferentially selected from octyldodecyl myristate, triethylhexanoin, dicaprylyl carbonate and isostearyl isostearate, caprylic/capric triglyceride;
butters such as shea butters preferentially butyrospernum parkii butter extract, shea butter ethyl esters, marketed under the names of LIPEX SHEASOFT, LIPEX SHEA-U, LIPEX SHEA, LIPEX SHEALIGHT, LIPEX SHEA TRIS or moringa butters preferentially moringa oil/hydrogenated moringa oil esters;
waxes preferentially selected from Acacia decurrens flower wax, Helianthus annuus cera seed wax, C10-18 triglycerides;
vegetable oils;
phytosqualane
alkanes preferentially selected from undecane and tridecane.

Preferably, the formulation according to the invention comprises, in addition to the composition according to the invention, at least one emollient agent having a content of between 0.1 and 30%, preferably between 0.5 and 10% by total weight of the formulation.

When the formulation according to the invention comprises at least one gelling and/or thickening agent, at least one gelling and/or thickening agent is selected from:
cellulose derivatives preferentially selected from gums of plant origin such as guar, carob, alginates, carrageenans, pectins, gums of microbial origin such as xanthan,
clays such as laponite
homo- and copolymers, whether crosslinked or not, hydrophilic or amphiphilic
acryloylmethylpropane sulfonic acid (AMPS) and/or acrylamide and/or acrylic acid and/or acrylic acid salts or esters (marketed under the names ARISTOFLEX AVC, AristoflexAVS, Aristoflex HMB, SIMULGEL NS, Simulgel EG, Simulgel 600, Simulgel 800, Pemulen, carbopol, Sepiplus 400, Seppimax zen, Sepiplus S, COSMEDIA SP).

Preferably, the formulation according to the invention comprises, in addition to the composition according to the invention, at least one gelling and/or thickening agent having a content of between 0.1 and 10% by total weight of the formulation.

When the formulation according to the invention comprises at least one surfactant, at least one surfactant is selected from:
anionic surfactants such as isethionates, taurates, sarcosinates, glycinates, glutamates, phosphates (C20-22 alkyl phosphate marketed under the name SENSANOV WR);
amphoteric surfactants such as betaine derivatives, amphoacetates;
non-ionic surfactants such as polyglycerol derivatives, sugar derivatives such as glucoside or xyloside derivatives (marketed under the names MONTANOV 68, MONTANOV 202, Montanov 82, MONTANOV L, EASYNOV), lecithins.

Preferably, the formulation according to the invention comprises, in addition to the composition according to the invention, at least one surfactant having a content of between 0.1 and 15%, preferably between 0.5 and 10% by total weight of the formulation.

When the formulation according to the invention comprises at least one film-forming polymer, at least one film-forming polymer is selected from:
synthetic polymers such as radical-like or polycondensate-like polymers,
polymers of natural origin and mixtures thereof, preferentially acrylic polymers, polyhydroxyalkanoates (PHAs), polyurethanes, polyesters, polyamides, polyureas and cellulose polymers such as nitrocellulose.

Preferably, the formulation according to the invention comprises, in addition to the composition according to the invention, at least one film-forming polymer having a content of between 0.5 and 20% by total weight of the formulation.

Advantageously, the film-forming polymer improves the hold and non-transfer properties of the formulation according to the invention, notably in make-up formulations.

According to one embodiment of the invention, the formulation comprises, in addition to the composition according to the invention, at least one non-volatile oil or organic solvent selected from non-volatile hydrocarbon and/or silicone oils.

When the composition comprises at least one oil or non-volatile organic solvent, it has a content of between 0.01 and 30 wt. %, preferentially between 0.1 and 25%, notably between 0.1 and 20% relative to the total weight of the formulation.

Preferably, the formulation according to the invention comprises, in addition to the composition according to the invention, at least one non-volatile organic oil or solvent having a content of between 0.01 and 30 wt. %, preferentially between 0.1 and 25%, notably between 0.1 and 20% relative to the total weight of the formulation.

According to another embodiment, the formulation according to the invention comprises, in addition to the composition according to the invention, at least one wax having a content of between 0.5 and 20%, preferably between 1 and 10% by total weight of the formulation.

When the formulation according to the invention comprises at least one filler, at least one filler is selected from talc, mica, silica, kaolin, boron nitride, starch, octenyl succinic anhydride modified starch, polyamides, silicone resins, silicone elastomer powders and acrylic polymer powders such as polymethyl methacrylate. The fillers used in the formulation according to the invention can notably consist of several layers of different chemical nature and/or physical form and notably be in the form of lamellae coated with spherical fillers. They can be modified using various surface treatments.

According to one embodiment, the formulation according to the invention comprises, in addition to the composition according to the invention, at least one coloring matter selected from a pigment or a mother-of-pearl.

When the formulation according to the invention comprises at least one coloring matter comprising a pigment, the pigment may be white or colored, mineral and/or organic, coated or uncoated.

Said pigment is preferentially selected from zirconium, zinc or cerium oxides, as well as iron or chromium oxides, manganese violet, ultramarine blue, chromium hydrate and ferric blue, carbon black, D & C like pigments, and lacquers based on cochineal carmine, barium, strontium, calcium or aluminum.

Pearlescent pigments can be preferentially selected from white pearlescent pigments such as mica coated with titanium or bismuth oxychloride, colored pearlescent pigments such as titanium mica with iron oxides, titanium mica with notably ferric blue or chromium oxide, titanium mica with an organic pigment of the aforementioned type as well as pearlescent pigments based on bismuth oxychloride.

Other additives usually used in cosmetics may also be present in the formulation according to the invention, notably preservatives, antioxidants or fragrances well known in the technical field.

A person skilled in the art is able to select from all of these possible additives both the nature and the quantity of any additives to be added to the composition, such that said composition retains all its properties.

According to another object, the invention relates to a composition having a clarity defined by an L* parameter greater than 90, an opacity greater than 80%, comprising the mixture of bismuth oxychloride, boron nitride and at least one other inorganic compound.

### Process

Also, according to another aspect, the object of the invention is a method for manufacturing a composition according to the invention comprising at least the implementation of the following steps:
a) Selecting each compound constituting the composition according to the invention: bismuth oxychloride, boron nitride and at least one other inorganic compound
b) Mixing the compounds selected in step a) by successive addition; and
c) Recovering a water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

The compounds from step a) are different inorganic compounds:
at least bismuth oxychloride and boron nitride; and
at least one other inorganic compound selected from: iron oxide, zinc oxide, cerium oxide, calcium carbonate, tricalcium phosphate, calcium sulfate, zinc stearate, magnesium carbonate, zinc sulfate, magnesium stearate, barium sulfate, zinc sulfide, aluminum powder, calcined clay, tricalcium phosphate, kaolin and combinations thereof.

The mixing in step b) is carried out by convection, diffusion or mechanical dispersion, preferentially the mixing in step b) is carried out using a powder mixer.

Advantageously, the powder mixer allows the composition according to the invention to be mixed without crushing the composition against the walls.

Preferably, the mixing in step b) is carried out in a powder mixer at a speed of between 1 and 20 rpm, preferentially between 1 and 10 rpm, even more preferentially at 10 rpm.

Advantageously, the mixing speed in step b) makes it possible to obtain a homogeneous mixture.

Preferentially, the mixing in step b) is carried out for between 1 and 10 hours, preferentially from 1 to 5 hours, even more preferentially 2 hours.

According to a particularly preferred embodiment, the mixing in step b) is carried out using a powder mixer at a speed of 5 rpm for 2 hours.

Advantageously, the mixing in step b) makes it possible to obtain a homogeneous composition.

According to one embodiment, the method according to the invention comprises a step d) of surface treatment. The surface treatment step, also known as coating, can be hydrophobic, hydrophilic, lipophobic or lipophilic.

Surface treatment step d) can be carried out with:
a silicone derivative preferably selected from Triethoxycaprylylsilane, Dimethicone and Methicone;
a fatty acid ester derivative preferably selected from stearoyl glutamic acid, polyhydroxystearic acid, hydrogenated olive oil stearyl ester, magnesium myristate, Jojoba ester, lecithin, hydrogenated lecithin, aloe vera oil extract and avocado oil;
waxes preferably selected from carnauba wax, polyethylene wax or olefin-like waxes;
silane derivatives, preferably selected from methoxy PEG-10 propyltrimethoxysilane;
silica;
phytic acid, inositol dihydrogen phosphate ester;
metallic salts of fatty acids;
metal alcoholates;
polysaccharides;
alkyl titanate;
natural or mineral oils;
amino acids or amino acid derivatives such as lauroyl lysine alone or combined with aluminum hydroxide;
a fatty acid stearic acid derivative, preferably isopropyl titanium triisostearate; and
a fluorosilane, preferably perfluorohexylethyl triethoxysilane.

A person skilled in the art can select the type of surface treatment based on the nature and the intended use of the formulation, notably when it is for cosmetic use.

Thus, the method according to the invention makes it possible to obtain a water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

### Uses

According to a final aspect, the invention relates to the use of a composition according to the invention in formulations such as inks, paints, plastics, cosmetic product formulations and medicines.

Preferentially, the composition according to the invention represents from 0.1 to 20 wt. % relative to the total weight of a formulation such as an ink, a paint, a plastic, a cosmetic product and medicine formulation.

In particular, the invention is aimed at the use of such a white composition as a TiO2 substituent, in such cosmetic formulations.

According to another embodiment, the invention is aimed at the use of the composition according to the invention in solvent-based paints or inks.

The composition according to the invention can be used notably in paints selected from the family of polyurethanes, acrylics or epoxides.

The composition according to the invention can also notably be used in inks selected from the family of epoxies, acrylics, naphtha solvents, nitrocelluloses, alkyds, polyurethanes, polyamides, ketones or polyesters.

Advantageously, the composition according to the invention can replace TiO2 with a clarity parameter equivalent to that of TiO2.

### EXAMPLES

Composition example according to the invention:

### Example 1

A composition according to the invention (reference 1) was produced according to the following protocol:

Reference 1: The composition according to the invention (reference 1) consists of:
bismuth oxychloride and boron nitride in a 0.13:1 ratio; and
zinc oxide representing less than 15% of the total mass of the composition.

Measurements of the L* parameter and opacity were carried out and these measurements were compared with commercial compositions targeting the same properties as well as with TiO2 commonly used in cosmetic compositions to date:

Reference 2: W877 organophilic TiO2: inorganic pigment comprising aluminum hydroxide-coated titanium oxide rutile, marketed by Sensient.
Reference 3: NOVAWHITE® mica/Zn: inorganic pigment comprising a mixture of zinc oxide and mica, marketed by The Innovation Company.
Reference 4: NOVAWHITE® Zn/Bo: inorganic pigment comprising a mixture of zinc oxide and bismuth oxychloride, coated with a hydrogenated oil mixture, marketed by The Innovation Company.
Reference 5: BN PUHP 1109C: boron nitride marketed by Saint-Gobain

### Protocol for Assessing L* and Opacity:

### 1. Preparing the Sample

Disperse 20% by weight of the pigment to be tested in 80% by weight of MARASTAR SR 910 E21 resin
Homogenize the sample by manual stirring, using a spatula, for 5 minutes
Pass the resulting dispersion through the EXAKT 50i three-roll mill, 1-1 gap
Apply the film to the BYK contrast chart using a BYK Byko Drive XL automatic applicator and a bar 50
Allow the sample to air dry for 30 minutes at room temperature
Check that the dry thickness is 35+/−5 μm using a micrometer (Palmer)

### 2. Measuring L* and C on the Following Samples:

The L* parameter is determined by measuring with an Xrite CI62 colorimeter on a white background and black background
Opacity C is determined by measuring with an Xrite CI62 colorimeter on a white background and black background

TABLE 1

| Baseline | L* on black background | Opacity (%) |
|---|---|---|
| 1 | 92.21 | 92.04 |
| 2 | 95.39 | 95.82 |
| 3 | 70.75 | 55.53 |
| 4 | 73.39 | 54.67 |
| 5 | 31.09 | 58.07 |

Reference 1, which complies with the invention, has L* and opacity properties closest to those of reference 2, representing titanium dioxide commonly used in the field of cosmetic compositions. Its L* and opacity values are also much closer to those of titanium dioxide than those of references 3, 4 and 5 which have been proposed as alternatives to titanium dioxide. Finally, compared with bismuth oxychloride which has an L* of 85 and an opacity of 85%, and boron nitride which has an L* of 90 and an opacity of 86% respectively, the composition of reference 1 according to the invention demonstrates the synergistic effect of the combination of these 2 compounds on L* clarity and opacity values.

### Example 2: Cosmetic Formulation

A cosmetic foundation formulation comprising reference 1 (composition according to the invention) as previously described was produced according to the elements in the Table below:

TABLE 2

| | | |
|---|---|---|
| INCI name | Trade names | % |

Oily phase

| | | |
|---|---|---|
| OCTYL PALMITATE | 2 ETHYLHEXYL | 15.00 |
| | PALMITATE | |
| NEOPENTYL GLYCOL | DUB DONPG | 6.80 |
| DIETHYLHEXANOATE | | |
| CAPRYLIC/CAPRIC | DUB MCT 5545 | 8.00 |
| TRIGLYCERIDE | | |
| CYCLOMETHICONE | MIRASIL CM5 | 5.00 |
| PEG-30 | CITHROL DPHS-SO-(MV) | 3.00 |
| DIPOLYHYDROXYSTEARATE | | |
| CYCLOPENTASILOXANE & | BENTONE GEL VS5V | 2.00 |
| DISTEARDIMONIUM | | |
| HECTORITE & SD ALCOHOL | | |
| 40 | | |
| POLYGLYCERYL-3 | CERA BELLINA | 0.25 |
| BEESWAX & TOCOPHEROL | | |
| & HEXYL CINNAMAL & | | |
| BENZYL ALCOHOL | | |

Aqueous phase

| | | |
|---|---|---|
| WATER | Demineralized Water | 33.00 |
| DUB DONPG | GLYCERINE 4811 | 3.00 |
| BUTYLENE GLYCOL | 1,3 BUTYLENE GLYCOL | 6.00 |
| CAPRYLYL GLYCOL | DERMOSOFT OCTIOL | 0.25 |
| POTASSIUM SORBATE | POTASSIUM SORBATE | 0.45 |

Powder phase

| | | |
|---|---|---|
| TALC | MICRO ACE P-3 | 7.00 |
| Composition according to the | | 8.00 |
| invention (reference 1 of | | |
| example 1) | | |
| CI 77492 (IRON OXIDES) & | NAI-C339001-10 | 2.25 |
| DISODIUM STEAROYL | | |
| GLUTAMATE & ALUMINUM | | |
| HYDROXIDE | | |

The cosmetic formulation offers good coverage and hold, is non-sticky and pleasant to the touch.

### Example 3: Comparative Test of the Clarity Measured on a Black Background and the Opacity of Compositions Based on the Content of the Mixture of Bismuth Oxychloride and Boron Nitride

For this example, 5 compositions were used, namely:
Composition 1 (outside the scope of the invention) comprising a mixture of bismuth oxychloride and boron nitride representing 17% of the weight of the composition;
Composition 2 (outside the scope of the invention) comprising a mixture of bismuth oxychloride and boron nitride representing 10% of the weight of the composition;
Composition 3 (outside the scope of the invention) comprising a mixture of bismuth oxychloride and boron nitride representing 45% of the weight of the composition;
Composition 4 (outside the scope of the invention) comprising a mixture of bismuth oxychloride and boron nitride representing 30% of the weight of the composition; and
Composition 5 (invention) comprising a mixture of bismuth oxychloride and boron nitride representing 80% of the weight of the composition.

The characterization of the above-mentioned compositions is described in Table 3 below:

TABLE 3

| | Composition | Composition | Composition | Composition | Composition |
|---|---|---|---|---|---|
| | 1 | 2 | 3 | 4 | 5 |
| | Percentage by weight of the composition | | | | |
| Bismuth | 5 | 5 | 25 | 10 | 24 |
| oxychloride | | | | | |
| Zinc oxide | 83 | 90 | 55 | 70 | 20 |
| Boron nitride | 12 | 5 | 20 | 20 | 56 |
| Total | 100 | 100 | 100 | 100 | 100 |

For these 5 compositions, a measurement of the clarity measured on a black background defined by the L* parameter and the opacity was carried out. The results are described in Table 4 below:

TABLE 4

| | Composition | Composition | Composition | Composition | Composition |
|---|---|---|---|---|---|
| | 1 | 2 | 3 | 4 | 5 |
| L* on black | 86.46% | 85.97% | 87.01% | 85.65% | 91.5% |
| background | | | | | |
| Opacity | 79.42% | 78.33% | 80.37% | 77.57% | 89.9% |

These results show that it is necessary for the composition according to the invention to have a mixture of bismuth oxychloride and boron nitride representing at least 50 wt. % by weight of the composition to obtain satisfactory colorimetric parameters similar to those of TiO2.

### Example 4: Composition Examples According to the Invention

For this example, 3 compositions were used, namely:
Composition 6 (invention) comprising the mixture of bismuth oxychloride, boron nitride and zinc sulfide, wherein the mixture of bismuth oxychloride and boron nitride is 97 wt. % relative to the total weight of the composition;
Composition 7 (invention) comprising the mixture of bismuth oxychloride, boron nitride and barium sulfate, wherein the mixture of bismuth oxychloride and boron nitride is 95 wt. % relative to the total weight of the composition;
Composition 8 (invention) comprising the mixture of bismuth oxychloride, boron nitride and calcium carbonate, wherein the mixture of bismuth oxychloride and boron nitride is 95 wt. % relative to the total weight of the composition;

The characterization of the above-mentioned compositions is described in Table 5 below:

TABLE 5

| | Composition 6 | Composition 7 | Composition 8 |
|---|---|---|---|
| | Percentage by weight of the composition | | |
| Bismuth oxychloride | 48.5 | 10 | 5 |
| Zinc sulfide | 3 | — | — |
| Barium sulfate | — | 5 | |
| Calcium carbonate | — | | 5 |
| Boron nitride | 48.5 | 85 | 90 |
| Total | 100 | 100 | 100 |

For these 3 compositions, a measurement of the clarity measured on a black background defined by the L* parameter and the opacity was carried out. The results are described in Table 6 below:

TABLE 6

| | Composition 6 | Composition 7 | Composition 8 |
|---|---|---|---|
| L* on black | 93% | 90.02% | 90.22% |
| background | | | |
| Opacity | 92% | 87.4% | 87.2% |

1. A water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

2. The composition according to claim 1, characterized in that the composition has a clarity defined by an L* parameter greater than 88, an opacity greater than 82%.

3. The composition according to claim 1, characterized in that said at least one other inorganic compound is selected from: iron oxide, zinc oxide, cerium oxide, calcium carbonate, tricalcium phosphate, calcium sulfate, zinc stearate, magnesium carbonate, zinc sulfate, magnesium stearate, barium sulfate, zinc sulfide, aluminum powder, calcined clay, tricalcium phosphate, kaolin and combinations thereof.

4. The composition according to claim 1, characterized in that the at least one other inorganic compound is selected from zinc stearate, zinc sulfate, zinc oxide, zinc sulfide and combinations thereof.

5. The composition according to claim 1, characterized in that the composition has an oil absorption of less than 55%.

6. The composition according to claim 1, characterized in that it exhibits greater clarity and/or opacity than each of the compounds constituting the composition.

7. The composition according to claim 1, characterized in that the composition has a clarity defined by an L* parameter greater than or equal to 92 and an opacity greater than or equal to 91%.

8. The composition according to claim 1, characterized in that the composition also comprises a coating.

9. Use of a composition according to claim 1 in inks, paints, plastics and cosmetic product formulations, said composition representing from 0.1% to 20% by weight relative to the total weight of the formulation

10. A method for manufacturing a composition according to claim 1, characterized in that it comprises the following steps:
a) Selecting each compound constituting the composition: bismuth oxychloride, boron nitride and at least one other inorganic compound,
b) Mixing the compounds selected in step a) by successive addition; and
c) Recovering a water-insoluble white composition comprising the mixture of bismuth oxychloride, boron nitride and at least one other inorganic compound, wherein the mixture of bismuth oxychloride and boron nitride is at least 50 wt. % relative to the total weight of the composition.

11. The method for manufacturing a composition according to claim 12, characterized in that the mixing in step b) is carried out by mechanical dispersion, using a powder mixer at a speed of between 1 and 20 rpm, and for a period of 1 to 10 hours.
